(19) United States
(12) Patent Application Publication
(10) Pub. No.: US 2015/0307606 A1
(43) Pub. Date: Oct. 29, 2015

(54) LYOPHILIZED SPHERICAL PELLETS OF ANTI-IL-23 ANTIBODIES

(71) Applicants: Ashwin BASARKAR, (US); Akhilesh BHAMBHANI, (US); Ramesh KASHI, (US); Shona P. MEHTA, (US); MERCK SHARP & DOHME CORP., Rahway, NJ (US)

(72) Inventors: Ashwin Basarkar, Springfield, NJ (US); Akhilesh Bhambhani, Doylestown, PA (US); Ramesh Kashi, Warren, NJ (US); Shona P. Mehta, Westfield, NJ (US)

(21) Appl. No.: 14/650,605

(22) PCT Filed: Dec. 9, 2013
(86) PCT No.: PCT/US13/73830

§ 371 Date: Jun. 9, 2015

Related U.S. Application Data
(60) Provisional application No. 61/737,036, filed on Dec. 13, 2012.

(51) Int. Cl. C07K 16/24; F26B 5/06; A61K 9/19

(52) U.S. Cl. CPC C07K 16/244; A61K 9/19; F26B 5/06; C07K 2317/565; C07K 2317/94

(57) ABSTRACT

Methods for preparing lyophilized pellets of antibodies that specifically bind to human IL-23 are described. The pellets have a substantially spherical shape and are prepared by freezing droplets of a liquid composition of a desired biological material on a flat, solid surface, in particular, a surface that does not have any cavities, followed by lyophilizing the frozen droplets. These methods are useful for preparing lyophilized pellets having a high concentration of anti-IL-23 antibody, and which have a faster reconstitution time than lyophilized powder cakes prepared in vials. Also provided are improved formulations for use in preparing lyophilized forms of antibodies that specifically bind to human IL-23.

### FIELD OF THE INVENTION

The present invention relates to methods for preparing lyophilized pellets of antibodies that inhibit the activity of human IL-23, wherein the pellets are spherical in shape and have fast reconstitution times.

### BACKGROUND OF THE INVENTION

Interleukin-23 (IL-23) is a heterodimeric cytokine comprised of two subunits, p19 which is unique to IL-23, and p40, which is shared with IL-12. The p19 subunit is structurally related to IL-6, granulocyte-colony stimulating factor (G-CSF), and the p35 subunit of IL-12. IL-23 mediates signaling by binding to a heterodimeric receptor, comprised of IL-23R and IL-12β1, which is shared by the IL-12 receptor. A number of early studies demonstrated that the consequences of a genetic deficiency in p40 (p40 knockout mouse; p40KO mouse) were more severe than those found in a p35KO mouse. Some of these results were eventually explained by the discovery of IL-23, and the finding that the p40KO prevents expression of not only IL-12, but also of IL-23. See, e.g., Oppmann et al. (2000) Immunity 13:715-725; Wiekowski et al. (2001) J. Immunol. 166:7563-7570; Parham et al. (2002) J. Immunol. 168:5699-708; Frucht (2002) Sci STKE 2002, E1-E3; Elkins et al. (2002) Infection Immunity 70:1936-1948).

Recent studies, through the use of p40 KO mice, have shown that blockade of both IL-23 and IL-12 is an effective treatment for various inflammatory and autoimmune disorders. However, the blockade of IL-12 through p40 appears to have various systemic consequences such as increased susceptibility to opportunistic microbial infections. Bowman et al. (2006) Curr. Opin. Infect. Dis. 19:245. Accordingly, specific blockade of the p19 subunit of IL-23 is preferred in the treatment of human disease because it interferes with the activity of IL-23 without interfering with the activity of IL-12.

Therapeutic antibodies may be used to block cytokine activity. A significant limitation in using antibodies as a therapeutic agent in vivo is the immunogenicity of the antibodies. As most monoclonal antibodies are derived from non-human species, repeated use in humans results in the generation of an immune response against the therapeutic antibody. Such an immune response results in a loss of therapeutic efficacy at a minimum, and potentially a fatal anaphylactic response. Accordingly, antibodies of reduced immunogenicity in humans, such as humanized or fully human antibodies, are preferred for treatment of human subjects. Exemplary therapeutic antibodies to IL-23p19 are disclosed in U.S. Patent Application Publication No. 2007/0009526, and in International Patent Publication Nos. WO 2007/076524, WO 2007/024846, WO 2007/147019, and WO 2009/043933 the disclosures of which are hereby incorporated by reference in their entireties. Additional humanized anti-IL-23p19 antibodies are disclosed in commonly assigned applications published as International Patent Publication Nos. WO 2008/103432 and WO 2008/103473, and in commonly-assigned U.S. Patent Application Publication No. 2007/0048315, the disclosures of which are hereby incorporated by reference in their entireties.

Biological materials, such as therapeutic monoclonal antibodies, are frequently preserved by lyophilizing aliquots of a liquid composition containing the biological material. The lyophilization process involves freezing a liquid sample which is then subjected to a vacuum so that the ice in the frozen sample directly changes to water vapor or sublimes. After the removal of ice, the sample temperature is gradually increased (while still under vacuum) and water is desorbed from the remaining non-ice phase of the sample.

Lyophilized cakes of a biological material are prepared by aliquoting into a glass container a desired amount of the biological material, which is typically present in a buffered solution with appropriate stabilizers (i.e., a “formulation”) and then subjecting the glass container containing the biological material to steps of cooling, freezing, annealing, primary drying and secondary drying. The glass container containing the dried biological material is typically stored for long periods of time at room temperature or under refrigerated conditions. The dried formulation containing the biological material is typically reconstituted by adding a liquid, usually water, to the glass container. Glass containers used for lyophilizing biological materials intended for use as therapeutics and vaccines typically have included glass vials and dual chamber injection devices, in which one chamber contains the lyophilized cake and the other chamber contains the reconstituting liquid.

Methods of lyophilizing biological materials in the form of spherically shaped pellets, i.e., beads, have also been described. In these methods, individual samples of the biological material are frozen and dried prior to placing a desired number of the dried samples into a storage container such as a glass vial. Historically, these methods relied on either (a) dispensing an aliquot of a liquid composition containing the desired amount of a biological material into a container of a cryogen such as liquid nitrogen, which results in direct contact of the biological material with the cryogen and/or (b) dispensing an aliquot of a liquid composition containing the biological material into a cavity present on a chilled solid plate, where the cavity contains the aliquot until it is frozen. It should also be noted that the use of plates with machined cavities often requires use of an automated system for detachment of the pellets from the cavity wall. Furthermore, reliance on a cavity to contain the liquid aliquot results in a volume restriction on the size of the aliquot and resulting pellet. Another approach, which is referred to as the die and punch method and uses a closed mould and compressive force to obtain a frozen pellet, suffers from a complex assembly design, leakage of fluid formation from the cavity and sticking of pellet to either the die or the punch.

Antibodies for use in human subjects must be stored prior to use and transported to the point of administration. Reproducibly attaining a desired level of antibody drug in a subject requires that the drug be stored in a formulation that maintains the bioactivity of the drug. The need exists for formulations of anti-human IL-23p19 antibodies for use, e.g., in treatment of inflammatory, autoimmune, and proliferative disorders. Preferably, such formulations, including formulations of antibodies that specifically bind to the p19 subunit of human IL-23, will enable rapid lyophilization of antibodies into an improved lyophilized form that is stable and can be reconstituted quickly, e.g. for use in devices for self-administration where the patient himself or herself effects reconstitution just prior to administration.

### SUMMARY OF THE INVENTION

The present invention provides solutions to these needs in the art and more. In one aspect, the present invention relates to a method for preparing dried pellets (<5% moisture) of an antibody that acts as an antagonist of human IL-23, such as an antibody that binds to human IL-23 or its receptor, including an antibody that specifically binds to the p19 subunit of human the IL-23, or the IL-23R subunit of the human IL-23 receptor.

In some embodiments, the methods of the present invention comprise dispensing at least one liquid droplet having a substantially spherical shape onto a solid and flat surface (i.e., lacking any sample wells or cavity), freezing the droplet on the surface without contacting the droplet with a cryogenic substance and lyophilizing the frozen droplet to produce a dried pellet that is substantially spherical in shape. The method may be used in a high throughput mode to prepare multiple dried pellets by simultaneously dispensing the desired number of droplets onto the solid, flat surface, freezing the droplets and lyophilizing the frozen droplets. It has been surprisingly found that pellets prepared by the method of the invention from a liquid formulation having a high concentration of a biological material such as a protein therapeutic may be combined into a set of dried pellets that has a faster reconstituted time than a single lyophilized cake prepared by freezing and lyophilizing the same volume of the liquid formulation in a glass container.

In various embodiments, the liquid droplet comprising the biological material to be lyophilized is dispensed at a speed and at a gap that prevents freezing of any portion of the droplet in the tip, and that maintains the dispensed droplet in simultaneous contact with the top surface of the metal plate and the open end of the dispensing tip until the droplet touching the plate is frozen. Exemplary dispensing speeds (in ml/min) include ranges from about 3 to about 75, from about 5 to 75 (e.g. for a 250 μl liquid droplet), from about 3 to 60 (e.g. for a 100 μl liquid droplet), and from about 1 to 30 (e.g. for 20 and 50 μl liquid droplets).

In one embodiment of the invention, the solid, flat surface is the top surface of a metal plate which comprises a bottom surface that is in physical contact with a heat sink adapted to maintain the top surface of the metal plate at a temperature of −90° C. or below, such as below −150° C. or −180° C., or between about −180° C. and about −196° C. Because the top surface of the metal plate is well below the freezing point of the liquid formulation, the droplet freezes essentially instantaneously with the bottom surface of the droplet touching the top surface of the metal plate. The gap distance between the open end of the dispensing tip and the top surface of the plate can be, e.g., between 0.1 and 0.5 cm, 0.75 cm, or 1.0 cm. In some embodiments the heat sink comprises a plurality of metal fins having first and second ends, and arranged perpendicularly to the metal plate, with the first end of each fin touching the bottom surface of the metal plate and the second end immersed in liquid nitrogen.

In another embodiment, the solid, flat surface is hydrophobic and comprises the top surface of a thin film that is maintained above 0° C. during the dispensing step. The dispensed droplet is frozen by cooling the thin film to a temperature below the freezing temperature of the formulation.

In some embodiments of the present invention the liquid formulation used in the lyophilization procedure comprises a total solute concentration of at least 25% (wt/wt).

In another aspect, the invention relates to lyophilized spherical pellets of antibodies that act as antagonists of human IL-23, such as antibodies that bind to IL-23 or its receptor, including antibodies that specifically bind to the p19 subunit of IL-23 or the IL-23R subunit of IL-23 receptor, made by the methods of the present invention. In various embodiments the lyophilized spherical pellet has a reconstitution time in water at room temperature of less than 3, 2 or 1 minute. In yet another aspect, the invention relates to a container comprising one or more of these pellets.

In various embodiments, the method of the present invention is performed using a solution of an antibody selected from the group consisting of an anti-human IL-23p19 antibody, such as humanized antibody 13B8, including humanized 13B8-b, or anti-IL-23p40 antibodies such as ustekinumab or briakinumab, or variants of any of these three antibodies comprising the same CDR sequences, or comprising the same light chain and heavy chain variable domains. In related embodiments, the lyophilized spherical pellets of the present invention comprise these same antibodies or variants.

In another aspect, the invention relates to novel formulations of anti-IL-23p19 antibodies, e.g. antibody 13B8-b or variants thereof having the same CDRs, useful in preparing the lyophilized spherical pellets of the present invention. Antibody 13B8-b is described in U.S. Pat. No. 8,263,748. Light and heavy chains for humanized antibody 13B8-b are provided at SEQ ID NOs. 14 and 7, respectively. In one embodiment the formulation comprises: an antibody that specifically binds to the p19 subunit of human IL-23 at 50-120 mg/ml, e.g. 100 mg/ml; sucrose, such as about 12.5% sucrose, e.g. 12.5% sucrose; trehalose, such as about 12.5% trehalose, e.g. 12.5% trehalose; polysorbate 80, such as about 0.05% PS-80, e.g. 0.05% PS-80; and histidine buffer pH 6, such as about 10 mM histidine buffer, e.g. 10 mM histidine buffer. In further embodiments the formulation comprises: an antibody that specifically binds to the p19 subunit of human IL-23 at 50-120 mg/ml, e.g. 100 mg/ml; about 30% sucrose, 30% trehalose, or some combination of sucrose and trehalose totaling about 25% or 30%, e.g. 25% or 30% sucrose, 25% or 30% trehalose, or a combination of sucrose and trehalose totaling 25% or 30%, such as 12.5% sucrose plus 12.5% trehalose; along with polysorbate 80, such as about 0.05% PS-80, e.g. 0.05% PS-80; and histidine buffer pH 6, such as about 10 mM histidine buffer, e.g. 10 mM histidine buffer.

In another aspect, these same formulations are useful in preparing traditional, non-spherical lyophilized forms of anti-IL-23p19 antibodies, e.g. antibody 13B8-b or variants thereof having the same CDRs.

### BRIEF DESCRIPTION OF THE DRAWINGS

FIG. 1 illustrates the freezing of a dispensed liquid droplet according to one embodiment of the invention, in which the dispensed droplet is transiently bound on either side by the open end of the dispensing tip and the top surface of a metal plate that has a bottom surface in contact with a heat sink comprising a plurality of metal fins immersed in liquid nitrogen contained in a reservoir.

FIG. 2 is a photograph of frozen droplets prepared on a metal plate as illustrated in FIG. 1, wherein the top surface of the metal plate was maintained at a temperature of −190° C.

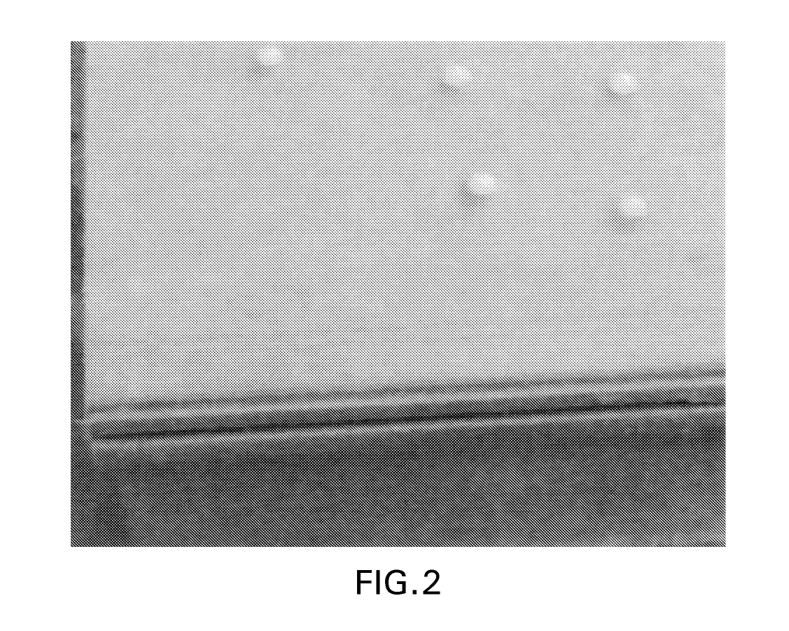

FIG. 3 is a photograph of dried pellets on a hydrophobic film prepared according to one embodiment of the invention.

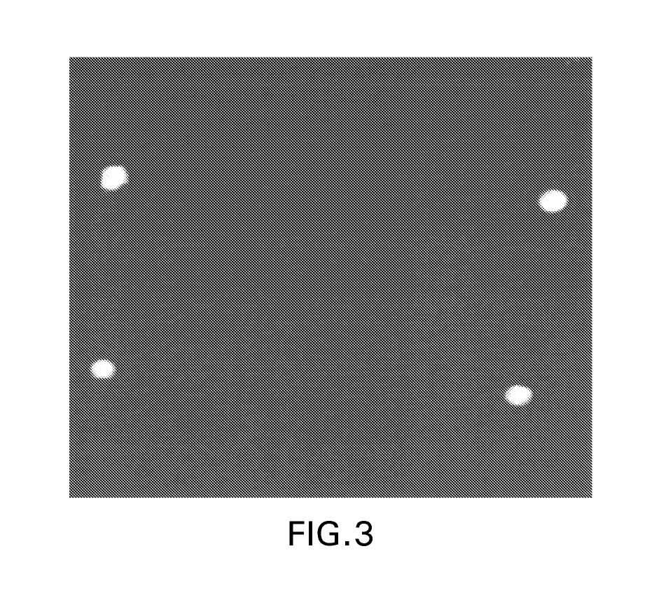

FIG. 4 is a photograph of 3 cc vials each containing 50 mg of a lyophilized antibody formulation that was prepared from a 100 mg/ml liquid antibody formulation. The left vial contains a lyophilized cake prepared by dispensing and lyophilizing 0.5 ml of the liquid antibody formulation in the vial; the middle vial contains 10 dried pellets, with each pellet prepared by dispensing 50 μl of the liquid antibody formulation onto a cold metal plate; and the right vial contains 5 pellets, with each pellet prepared by dispensing 100 μl of the liquid antibody formulation onto a cold metal plate.

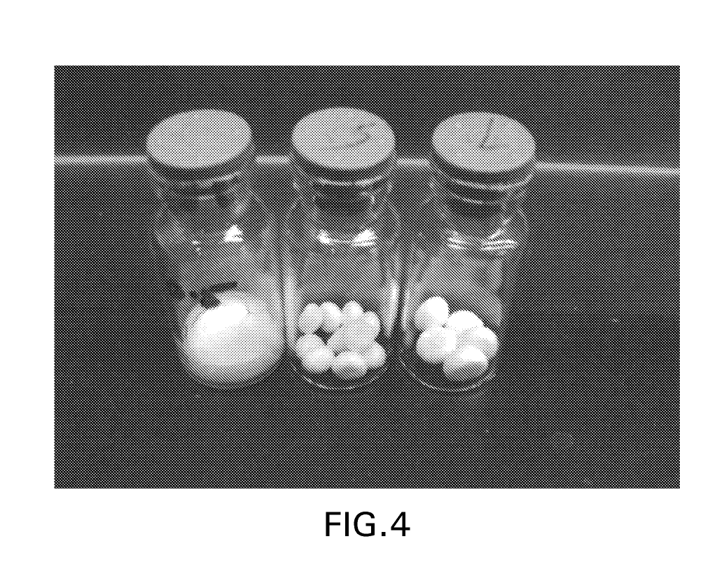

FIG. 5 is a photograph taken 10 min after adding water to four vials containing equivalent amounts (50 mg) of a lyophilized antibody formulation prepared from a 100 mg/ml liquid antibody formulation. The left vial contained a lyophilized cake prepared by dispensing and lyophilizing 0.5 ml of the liquid antibody formulation in the vial; the middle two vials contained 10 dried pellets, with each pellet prepared by dispensing 50 μl of the liquid antibody formulation onto a cold metal plate; and the right vial containing 5 pellets, with each pellet prepared by dispensing 100 μl of the liquid antibody formulation onto a cold metal plate.

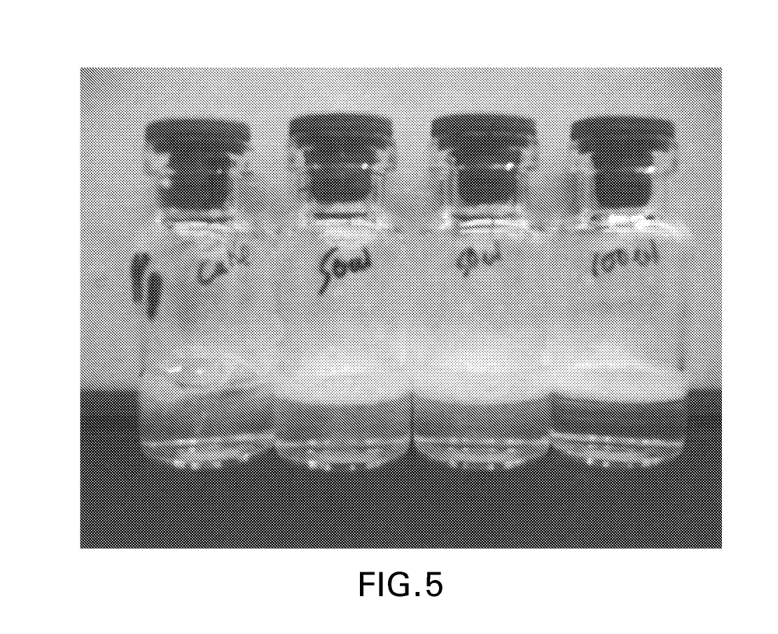

FIG. 6 presents the percentage of aggregates, as measured by high pressure size exclusion chromatography (HP-SEC), as a function of storage over 6 months, e.g. under accelerated degradation conditions (storage at 40° C.). Lyospheres of the present invention derived from a 25% “disaccharide formulation” (12.5% sucrose/12.5% trehalose, 0.05% polysorbate-80, 10 mM Histidine pH 6.0) show reduced accumulation of aggregates when compared with lyophilized cakes derived from a 7% sucrose formulation (7% sucrose, 0.05% polysorbate-80, 10 mM Histidine pH 6.0). See Example 5.

### DETAILED DESCRIPTION

As used herein, including the appended claims, the singular forms of words such as “a,” “an,” and “the,” include their corresponding plural references unless the context clearly dictates otherwise. Table 4 below provides a listing of sequence identifiers used in this application. All references cited herein are incorporated by reference to the same extent as if each individual publication, patent application, or patent, was specifically and individually indicated to be incorporated by reference. Citation of the references herein is not intended as an admission that any of the foregoing is pertinent prior art, nor does it constitute any admission as to the contents or date of these publications or documents.

As used herein with reference to the lyophilized pellets of the invention, the term “spherical pellet” is intended to refer to substantially spherical pellets, and does not require that such pellets be perfectly spherical to fall within the scope of the present invention. The shapes of the pellets of the present invention will be substantially spherical based on their formation from droplets of solution suspended between a dispensing tip and a flat surface, in which the bulk of the surface area of the droplets is determined by surface tension.

### I. Methods of Making Spherical Lyophilized Pellets of Biological Materials

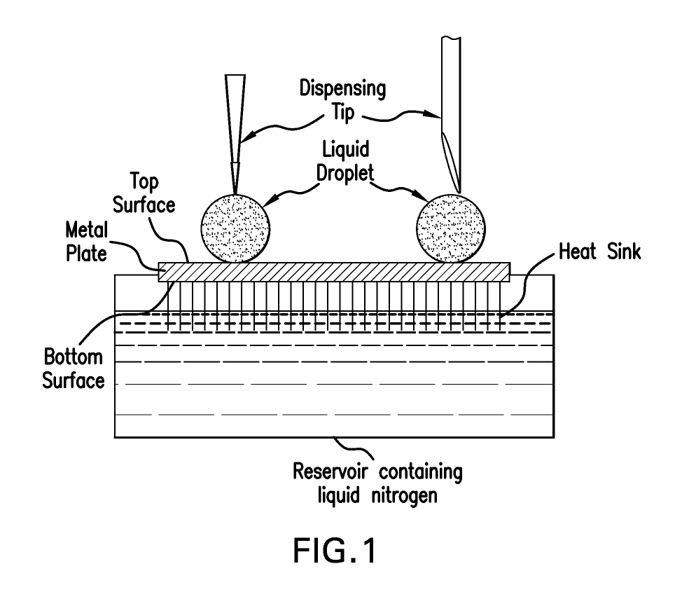

The method of making dried pellets of a biological material according to the invention comprises loading an aliquot of a liquid composition (such as a liquid protein formulation) comprising the biological material into a dispensing tip and dispensing the aliquot onto a solid, flat surface in such a way that the droplet remains intact while being dispensed. The term “solid, flat surface” means that there are no cavities or wells. Dispensing tips useful in the present invention include those with a round open end, and a pointed open end, as shown in FIG. 1. Multiple dried pellets may be prepared simultaneously by loading simultaneously the desired number of aliquots of the liquid composition into a multichannel pipettor.

In one embodiment, the solid, flat surface is the top surface of a metal plate and is maintained at a temperature of −90° C. or lower. In some embodiments of the invention, the temperature of the metal plate is −150° C. or lower, or −180° C. or lower. In other embodiments, the temperature of the plate is within a range of about −90° C. to about −130° C., about −110° C. to about −150° C., about −150° C. to about −195° C. or −180° C. to about −196° C. The metal plate comprises a conductive, inert metal such as gold, silver, stainless steel, aluminum or copper. In a preferred embodiment, the metal plate is comprised of aluminum. In another preferred embodiment, the plate is stainless steel. In some embodiments, the metal plate is rectangular in shape, and in one preferred embodiment, the dimensions of the rectangular plate are 10 inches long×7 inches wide×0.4 inches thick. Larger equipment is likely employed for large-scale production.

The cold temperature of the metal plate is maintained by placing the bottom surface of the metal plate in physical contact with a heat sink. In one preferred embodiment, the heat sink comprises a plurality of fins composed of a temperature conductive metal. In some embodiments, the fins are spaced about 0.25 inches apart along the bottom surface of the metal plate, with each fin having a length of at least about one inch. In an exemplary embodiment suitable for small scale production and optimization, a 10 inch×7 inch plate is used, and the heat sink preferably comprises thirty, one inch long fins.

The fins may be physically connected to the bottom of the metal plate using any of a multitude of approaches well-known in the art, for example, using metal screws, welding, gluing with a cryoglue. In such an embodiment, the term “bottom surface” means the surface of the plate that is physically connected to the plurality of fins. Alternatively, the metal plate and heat sink may be fabricated from a single metal block and in such a case, the skilled artisan will understand that the bottom surface of the metal plate and heat sink form part of the same functional feature and thereby in physical contact with each other.

An example of a heat sink that is fabricated from a single metal block, and useful in the present invention, is illustrated in FIG. 1. This plate comprises a plurality of metal fins having one end in physical contact with the bottom surface of the metal plate, which rests on top of a metal reservoir containing a liquid cryogen such as liquid nitrogen. Other liquid cryogens that may be used in the heat sink include liquid propane, isopentane/hexane mixtures, argon and HFE-7100. The metal fins and reservoir are preferably made of the same conductive metal as used for the plate. Similar heat sinks may be purchased commercially, e.g., from M&M Metals, 1305W Crosby Road, Carrollton, Tex.

In another embodiment, the solid, flat surface is hydrophobic and is maintained above 0° C. during the dispensing step, and preferably between 4° C. and 25° C. The hydrophobic surface may comprise a chemically inert plastic such as polytetrafluoroethylene (PTFE), polypropylene and the like. The hydrophobic surface may be bonded to a different material or simply comprise the top surface of a thin film made using the hydrophobic material (e.g., PTFE, polypropylene). To freeze the liquid droplet, the film containing the dispensed droplet is chilled to a temperature that is below the freezing point of the liquid composition comprising the biological material, and preferably to a temperature of about 5° C. to 25° C. below the freezing point.

It is important to maintain the liquid droplet intact during the dispensing step. When the droplet is dispensed onto a cold metal surface (i.e., −90° C. or lower), one way of accomplishing this is to dispense the droplet at a dispensing speed and at a distance between top surface and the bottom of the dispensing tip (the “gap distance”) that prevents the droplet from freezing while any portion of the droplet is still in the tip, and maintains the dispensed droplet in simultaneous contact with the top surface of the metal plate and the bottom of the dispensing tip, for example as shown in FIG. 1. This allows the droplet to freeze from the bottom up as it contacts the cold metal surface.

The dispensing speed and gap distance will depend upon the volume of the liquid droplet, and the shape of the open end of the dispensing tip, and may be readily determined experimentally. In preferred embodiments, the dispensing speed is within the range of about 3 ml/min to about 75 ml/min, about 5 ml/min to about 75 ml/min, about 3 ml/min to about 60 ml/min, about 20 ml/min to about 75 ml/min, about 20 ml/min to about 60 ml/min, and about 1 ml/min to about 30 ml/min. The dispensing time is the time required to dispense an aliquot of a given volume at a given dispensing speed. For a 250 μl bead, for example, this time could range from 0.2 second to 3.0 seconds. Similarly for 100 μl bead, for example, the dispensing time could range from 0.1 second to 2 seconds. Similarly for 50 μl bead, for example, the dispensing time could range from 0.1 second to 3 seconds. Similarly for 20 μl bead, for example, the dispensing time could range from 0.04 second to 1.2 seconds. In some embodiments, a suitable dispensing speed for preparing 50 and 20 μl droplets is 4.5 ml/min of a composition with low solute concentration (5%) and 9 ml/min for a composition with high solute (25%) concentration.

In an alternative embodiment, the gap distance (i.e., between the open end of the dispensing tip and the top surface) is high enough so that the dispensed drop is in contact only with the top surface of the cold metal plate. To maintain the intactness and spherical shape of the droplet, the temperature of the metal surface is maintained well below −150° C. to ensure instantaneous freezing of the liquid droplet as it touches the surface. The gap distance will depend on the volume of the dispensed aliquot, but is usually at least 1 cm.

When the liquid droplet is dispensed onto a hydrophobic surface, the droplet is typically maintained intact in a substantially spherical shape by choosing a volume for the aliquot that will remain intact as the droplet touches the surface.

In preferred embodiments, the dispensing tip or tips are connected to an automated dispensing unit capable of controlling the dispensing speed and the gap distance. Examples of automated dispensing units include the Biomek® FX Liquid Handling System and pipettors manufactured by Tecan.

In some embodiments, the method further comprises measuring the reconstitution time of the dried pellet. The term “reconstitution time” refers to the time that is required to completely dissolve a dried pellet, i.e., prepared according to the present invention, or a lyophilized cake to produce a reconstituted liquid formulation that is clear.

After the pellets are frozen, they are placed in a lyophilization chamber and lyophilized. The steps of a typical lyophilization cycle useful in the present invention include loading, annealing, freezing, and one or more drying steps. In some embodiments, the drying step(s) is performed above 0° C. A preferred lyophilization cycle will keep the drying droplet below the collapse temperature and produce a dried pellet of substantially the same shape and size of the frozen droplet, and having a moisture content of about 0.1% to about 10%, about 0.1% to about 6%, about 0.1% to about 3% or 0.5% to about 5%. Examples of lyophilization cycles are shown below. Parameters are presented as the target temperature for each step; the temperature change rate for reaching that target temperature; and the incubation time once the target temperature is reached. Drying steps also include the pressure during the incubation step.

### Lyophilization Parameters I

Load: −45° C.; 0.5° C./min; 15 min Annealing: −20° C.; 0.5° C./min; 60 min Freezing: −45° C.; 0.5° C./min; 75 min Primary Drying: 30° C.; 0.65° C./min; 1350 min @ 30 mTorr Secondary Drying: 30° C.; 0.65° C./min; 270 min @ 255 mTorr

### Lyophilization Parameters II

Load: −45° C.; 0.5° C./min; 15 min
Annealing: −20° C.; 0.5° C./min; 60 min
Freezing: −45° C.; 0.5° C./min; 75 min
Primary Drying: 15° C.; 0.65° C./min; 1590 min @ 30 mTorr
Secondary Drying: 30° C.; 0.65° C./min; 300 min @ 255 mTorr

### Lyophilization Parameters III

Load: −45° C.; 0.5° C./min; 15 min
Annealing: −20° C.; 0.5° C./min; 60 min
Freezing: −45° C.; 0.5° C./min; 75 min
Primary Drying: 15° C.; 0.65° C./min; 28 hr @ 30 mTorr
Secondary Drying: 15° C.; 0.65° C./min; 5 hr @ 210 mTorr

After completion of lyophilization, the dried pellets may be placed in a container for bulk storage, or aliquoted into desired end-use container. Bulk storage containers include, e.g., plastic trays, metal trays, bottles, foil bags, and the like. The desired end-use container may be configured to receive a liquid for reconstitution directly in the container, e.g., a vial, or commercially available dual chamber containers, such as a dual-chamber cartridge pen device, dual chamber foil packet, a plastic tube with two or more chambers and designed to readily mix two or more components immediately before administration of the therapeutic or vaccine in the pellet. Alternatively, the end-use container may be adapted to allow removal of a desired number of pellets, e.g., such as a bead dispenser, and the removed pellets are then reconstituted with liquid in a separate container.

The method of the present invention may be utilized to prepare dried pellets of a variety of biological materials, including therapeutic proteins such as cytokines, enzymes and antibodies, as well as antigenic substances used in vaccines, such as peptides and proteins. The biological material is typically in a liquid composition that also contains one or more components that confer stability on the biological material during storage of the liquid formulation, as well as during and after the freezing and lyophilization steps. This liquid composition is also referred to herein as a “liquid formulation, “pharmaceutical composition,” “vaccine composition,” and “vaccine formulation”. Additional components that may be included as appropriate include pharmaceutically acceptable excipients, additives, diluents, buffers, sugars, amino acids (such as glycine, glutamine, asparagine, arginine or lysine), chelating agents, surfactants, polyols, bulking agents, stabilizers, cryoprotectants, lyoprotectants, solubilizers, emulsifiers, salts, adjuvants, tonicity enhancing agents (such as alkali metal halides, preferably sodium or potassium chloride, mannitol, sorbitol), delivery vehicles and anti-microbial preservatives. Acceptable formulation components for pharmaceutical preparations are nontoxic to recipients at the dosages and concentrations employed.

In some embodiments, the total excipient concentration in the composition used to prepare the pellets comprises 50% or less on a weight by weight basis (w/w) of excipients that have plasticizing effects, such as glycerol and sorbitol. Such excipients result in dried pellets that are fragile or spongy, which are undesirable characteristics for subsequent processing operations. The skilled artisan can readily identify other excipients that have plasticizing effects. In other embodiments, the pellets are prepared from compositions having at least 5% solute concentration w/w.

The inclusion of cationic polymers, such as polybrene, that are typically used in cell culture for manufacturing virus antigens and proteins, should be avoided as the inventors herein have surprisingly discovered that even small amounts (e.g., a 5 microgram concentration) of polybrene in the composition results in pellets that fracture during or after freezing.

The method of the present invention is particularly useful for preparing dried pellets from liquid formulations having a high concentration of a therapeutic antibody, e.g. 50 mg/ml or more, and that has a reconstitution time of less than 3 minutes, preferably less than 2 min. The dried pellet is typically stable for at least 1 month at room temperature (e.g., 25° C.), and preferably at least 6 months at room temperature (e.g., 25° C.). Upon reconstitution, the formulation is suitable for parenteral administration such as intravenous, intramuscular, intraperitoneal or subcutaneous injection.

The method of the present invention is also particularly useful for preparing dried spherical pellets from compositions having a high solute concentration, e.g., concentrations above 20%. Such compositions may have high concentrations of sugars and other stabilizers, e.g., sucrose, trehalose, sucrose/trehalose mixtures, mannitol, dextrose, dextran and mixtures of such sugars. Compositions with a high solute concentration are not typically employed in products lyophilized in vials due to difficulty in achieving a satisfactory dried product with reasonable lyophilization cycles. However, as demonstrated below, frozen spherical droplets using the method described herein may be prepared from different types of compositions, including compositions with a low or high solute concentration, and dried using shorter lyophilization cycles than if done in vials.

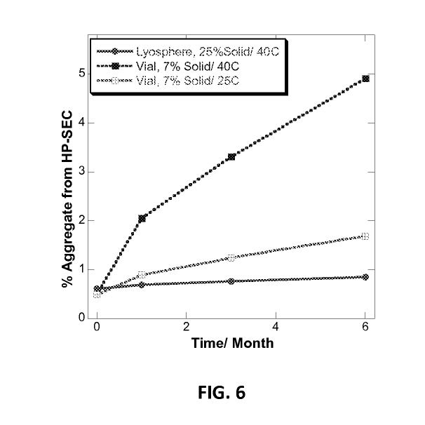

In some embodiments, a high concentration disaccharide formulation is used to form the spherical lyophilized pellets of the present invention. FIG. 6 provides the results of experiments demonstrating that a 25% disaccharide formulation of anti-human IL-23p19 mAb hum 13b8 exhibits superior stability as compared with standard lyophilized cakes made from a 7% sucrose formulation. As a matter of convenience, the 25% disaccharide lyospheres exhibit significantly faster lyophilization and reconstitution times when compared with traditional lyophilized cakes made with the 7% sucrose formulation. More significantly, after 6 months under accelerated degradation conditions (40° C.) the 25% disaccharide lyospheres show approximately 6-fold lower aggregate accumulation, and significantly less aggregation even than the 7% formulation-derived lyophilized cake stored at 25° C. These results suggest that lyospheres of the present invention, in particular those made from high concentration disaccharide formulations, such as 25% disaccharide, might make it possible to store therapeutic antibodies at room temperature, eliminating the need for cold chain transportation and storage. Such convenient transport and storage would be of significant benefit, particularly in environments where cold chain is not readily available, and could avoid product loss than might otherwise results from temperature excursions during packaging and distribution.

The dried pellets prepared by the method of the present invention can be easily integrated into a variety of dosage sizes by choosing the volume of the droplet used to prepare each pellet and the number of pellets added to a single or multiple dosage container or delivery device. Also, the invention readily enables the preparation of combination therapeutic or immunogenic products, in which dried pellets comprising one biological material are combined in a single container with dried pellets comprising a different biological material. For example, pellets prepared from different antigen compositions, such as measles, mumps, rubella, and varicella, may be combined in a single container to obtain a multi-component vaccine. This allows the different antigens to remain separate until reconstitution, which can increase shelf-life of the vaccine. Similarly, combination products may contain separate antigen-comprising pellets and adjuvant-comprising pellets. Another example would be a combination of pellets comprising a protein with pellets comprising a peptide.

### II. Lyophilized Spherical Pellets of IL-23 Antagonist Antibodies

The formulations and methods of the present invention can be used to prepare lyophilized spherical pellets of antibodies to human IL-23, e.g. for use in treatment of autoimmune, inflammatory and proliferative disorders. Interleukin-23 (IL-23) is a heterodimeric cytokine comprised of two subunits, p19 which is unique to IL-23, and p40, which is shared with IL-12. The p19 subunit is structurally related to IL-6, granulocyte-colony stimulating factor (G-CSF), and the p35 subunit of IL-12. IL-23 mediates signaling by binding to a heterodimeric receptor, comprised of IL-23R and IL-12β1, which is shared by the IL-12 receptor. A number of early studies demonstrated that the consequences of a genetic deficiency in p40 (p40 knockout mouse; p40KO mouse) were more severe than those found in a p35KO mouse. Some of these results were eventually explained by the discovery of IL-23, and the finding that the p40KO prevents expression of not only IL-12, but also of IL-23 (see, e.g., Oppmann et al. (2000) Immunity 13:715-725; Wiekowski et al. (2001) J. Immunol. 166:7563-7570; Parham et al. (2002) J. Immunol. 168:5699-708; Frucht (2002) Sci STKE 2002, E1-E3; Elkins et al. (2002) Infection Immunity 70:1936-1948).

Recent studies, through the use of p40 KO mice, have shown that blockade of both IL-23 and IL-12 is an effective treatment for various inflammatory and autoimmune disorders. IL-23 is known to play a central role in psoriasis, and the IL-23/IL-12 antagonist antibody ustekinumab (anti-IL-12/23p40 mAb) has been approved in the U.S. and Europe for the treatment of psoriasis. However, the blockade of IL-12 through p40 appears to have various systemic consequences such as increased susceptibility to opportunistic microbial infections. Bowman et al. (2006) Curr. Opin. Infect. Dis. 19:245.

Therapeutic antibodies may be used to block cytokine activity. The most significant limitation in using antibodies as a therapeutic agent in vivo is the immunogenicity of the antibodies. As most monoclonal antibodies are derived from rodents, repeated use in humans results in the generation of an immune response against the therapeutic antibody. Such an immune response results in a loss of therapeutic efficacy at a minimum and a potential fatal anaphylactic response at a maximum. Initial efforts to reduce the immunogenicity of rodent antibodies involved the production of chimeric antibodies, in which mouse variable regions were fused with human constant regions. Liu et al. (1987) Proc. Natl. Acad. Sci. USA 84:3439-43. However, mice injected with hybrids of human variable regions and mouse constant regions develop a strong anti-antibody response directed against the human variable region, suggesting that the retention of the entire rodent Fv region in such chimeric antibodies may still result in unwanted immunogenicity in patients.

It is generally believed that complementarity determining region (CDR) loops of variable domains comprise the binding site of antibody molecules. Therefore, the grafting of rodent CDR loops onto human frameworks (i.e., humanization) was attempted to further minimize rodent sequences. Jones et al. (1986) Nature 321:522; Verhoeyen et al. (1988) Science 239:1534. However, CDR loop exchanges still do not uniformly result in an antibody with the same binding properties as the antibody of origin. Changes in framework residues (FR), residues involved in CDR loop support, in humanized antibodies also are required to preserve antigen binding affinity. Kabat et al. (1991) J. Immunol. 147:1709. While the use of CDR grafting and framework residue preservation in a number of humanized antibody constructs has been reported, it is difficult to predict if a particular sequence will result in the antibody with the desired binding, and sometimes biological, properties. See, e.g., Queen et al. (1989) Proc. Natl. Acad. Sci. USA 86:10029, Gorman et al. (1991) Proc. Natl. Acad. Sci. USA 88:4181, and Hodgson (1991) Biotechnology (NY) 9:421-5. Moreover, most prior studies used different human sequences for animal light and heavy variable sequences, rendering the predictive nature of such studies questionable. Sequences of known antibodies have been used or, more typically, those of antibodies having known X-ray structures, antibodies NEW and KOL. See, e.g., Jones et al., supra; Verhoeyen et al., supra; and Gorman et al., supra. Exact sequence information has been reported for a few humanized constructs.

### III. Exemplary IL-23 Antagonist Antibodies

In some embodiments anti-IL-23 antibody does not antagonize the activity of IL-12, i.e. the antibody is IL-23-specific. Such IL-23-specific antibodies include antibodies that bind specifically to the p19 subunit of IL-23, rather than the p40 subunit, since the p19 subunit is specific to IL-23 (p19+p40) whereas the p40 subunit is shared with IL-12 (p35+p40). Exemplary IL-23-specific antibodies that bind to p19 are disclosed at WO 2008/103432, US 2007/0048315 and WO 2008/103473 (to Schering Corp.); U.S. Pat. No. 7,491,391, U.S. Pat. No. 7,935,344 and EP 1971366 A2 (to Centocor Ortho Biotech, Inc.); U.S. Pat. No. 7,872,102 (to Eli Lilly and Co.); WO 2007/147019, WO 2008/134659 and WO 2009/082624 (to Zymogenetics); US 2009/0311253 (to Abbott Bioresearch); and US 2009/0123479 and WO 2010/115786 (to Glaxo SmithKline), the disclosures of which are hereby incorporated by reference in their entireties.

Anti-IL-23p19 antibodies that may be suitable for use in the methods of the present invention also include, but are not limited to, Merck's SCH 900222/MK-3222; Eli Lilly's LY2525623, and Centocor's CNTO 1959, all of which have entered human clinical trials. Specifically, the sequences of SEQ ID NOs: 48 and 52 (heavy chain variable domains), 57 (light chain variable domain), 28-37-40 (light chain CDRs 1-2-3, respectively) and 3-8-19 (light chain CDRs 1-2-3, respectively) of EP 1937721 B1 (to Eli Lilly and Company) are hereby incorporated by reference. In addition, the sequences of SEQ ID NOs: 106 (heavy chain variable domain), 116 (light chain variable domain), 50-56-73 (light chain CDRs 1-2-3, respectively) and 5-20-44 (light chain CDRs 1-2-3, respectively) of U.S. Pat. No. 7,935,344 (to Centocor) are also hereby incorporated by reference.

In some embodiments, the anti-IL-23p19 antibodies, or antigen binding fragments thereof, are based on antibody 13B8 of commonly assigned WO 2008/103432, the disclosure of which is hereby incorporated by reference in its entirety. The anti-human IL-23p19 antibody may comprise one, two, three, four, five or six of the CDR sequences, or the heavy and light chain variable domains, of the humanized antibodies disclosed in commonly assigned WO 2008/103432, for example antibodies hum13B8-a, -b or -c. In another embodiment the anti-human IL-23p19 antibody competes with antibody hum13B8-a, -b or -c for binding to human IL-23. In another embodiment the anti-human IL-23p19 antibody binds to the same epitope on human IL-23 as hum13B8-a, -b or -c.

A hybridoma expressing antibody 13B8 was deposited pursuant to the Budapest Treaty with American Type Culture Collection (ATCC—Manassas, Va., USA) on Aug. 17, 2006 under Accession Number PTA-7803. All restrictions on the accessibility of this deposit will be irrevocably removed upon the granting of a U.S. patent based on the present application. In other embodiments, the anti-human IL-23p19 antibody is able to block binding of human IL-23p19 to the antibody produced by the hybridoma deposited with accession number PTA-7803 in a cross-blocking assay. In yet further embodiments, the anti-human IL-23p19 antibody binds to the same epitope as the antibody produced by the hybridoma deposited with ATCC under accession number PTA-7803. In still further embodiments, the anti-human IL-23p19 antibody comprises the same CDR sequences as the antibody produced by the hybridoma deposited with ATCC with accession number PTA-7803.

IL-23-specific antibodies also include antibodies that bind specifically to IL-23p40 but not IL-12p40 (U.S. Pat. No. 7,247,711 to Centocor) or antibodies that make contacts with both the p19 and p40 subunits of IL-23 (WO 2011/056600 to Amgen, Inc.).

IL-23 activity may also be blocked using antibodies that specifically bind to the IL-23R subunit of the IL-23 receptor complex (IL-23R+IL-12Rβ1), rather than the IL-12Rβ1 subunit that is shared with the IL-12 receptor (IL-12Rβ1+IL-12Rβ2). Exemplary anti-human IL-23R antibodies are disclosed at WO 2008/106134 and WO 2010/027767 (to Schering Corp.).

In other embodiments, the IL-23 antibody is a non-specific IL-23 antibody Exemplary non-specific IL-23 antibodies include antibodies that bind to the p40 subunit of IL-23 and IL-12, such as ustekinumab (CNTO 1275) and briakinumab (ABT-874, J-695). Ustekinumab is marketed by Centocor for the treatment of psoriasis, and is described at U.S. Pat. No. 6,902,734 and U.S. Pat. No. 7,166,285 (to Centocor, Inc.), the disclosures of which are hereby incorporated by reference in their entireties. Specifically, the sequences of SEQ ID NOs: 7 (heavy chain variable domain) and 8 (light chain variable domain), of U.S. Pat. No. 6,902,734 are hereby incorporated by reference. SEQ ID NOs: 4-5-6 and 1-2-3 of U.S. Pat. No. 6,902,734 are also incorporated by reference. Sequences for ustekinumab are also provided at SEQ ID NOs: 51-60 of the sequence listing of the present application. Briakinumab was developed by Abbott, and is described at U.S. Pat. No. 6,914,128 and U.S. Pat. No. 7,504,485, the disclosures of which are hereby incorporated by reference in their entireties. Specifically, the sequences of SEQ ID NOs: 31 (heavy chain variable domain), 32 (light chain variable domain) SEQ ID NOs; 30-28-26 (light chain CDRs 1-2-3, respectively) and 29-27-25 (heavy chain CDRs 1-2-3, respectively) of U.S. Pat. No. 6,914,128 are hereby incorporated by reference. Sequences for briakinumab are also provided at SEQ ID NOs: 61-70 of the sequence listing of the present application.

Further exemplary non-specific IL-23 antagonist antibodies that bind to the p40 subunit of IL-23 and IL-12 are disclosed at Clarke et al. (2010) mAbs 2:1-11 (Cephalon Australia, Pty., Ltd.). FM202 (Femta Pharmaceuticals) is also a monoclonal antibody that binds to the p40 subunit of both IL-12 and IL-23, as are the antibodies disclosed at WO 2010/017598 (Arana Therapeutics, Ltd.). Still further exemplary non-specific IL-23 antagonists include antibodies that bind to the IL-12Rβ1 subunit of both the IL-12 and IL-23 receptor complexes (WO 2010/112458 to Novartis AG).

In some embodiments involving anti-IL-23p19 antibodies, of antigen binding fragments thereof, amino acid sequence variants of the human or humanized anti-IL-23 antibody will have an amino acid sequence having at least 75% amino acid sequence identity with the original human or humanized antibody amino acid sequences of either the heavy or the light chain more preferably at least 80%, more preferably at least 85%, more preferably at least 90%, and most preferably at least 95, 98, or 99%. Identity or homology with respect to this sequence is defined herein as the percentage of amino acid residues in the candidate sequence that are identical with the human or humanized anti-IL-23 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

The human or humanized antibody can be selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Variants of the IgG isotypes are also contemplated. The human or humanized antibody may comprise sequences from more than one class or isotype. Optimization of the necessary constant domain sequences to generate the desired biologic activity is readily achieved by screening the antibodies in the biological assays described below.

Likewise, either class of light chain can be used in the compositions and methods herein. Specifically, kappa, lambda, or variants thereof are useful in the present compositions and methods.

For some embodiments involving humanized anti-IL-23p19 antibodies, any suitable portion of the CDR sequences from the non-human antibody can be used. The CDR sequences can be mutagenized by substitution, insertion or deletion of at least one residue such that the CDR sequence is distinct from the human and non-human antibody sequence employed. It is contemplated that such mutations would be minimal. Typically, at least 75% of the humanized antibody residues will correspond to those of the non-human CDR residues, more often 90%, and most preferably greater than 95%.

For some embodiments involving humanized anti-IL-23p19 antibodies, any suitable portion of the FR sequences from the human antibody can be used. The FR sequences can be mutagenized by substitution, insertion or deletion of at least one residue such that the FR sequence is distinct from the human and non-human antibody sequence employed. It is contemplated that such mutations would be minimal. Typically, at least 75% of the humanized antibody residues will correspond to those of the human FR residues, more often 90%, and most preferably greater than 95, 98, or 99%.

CDR and FR residues are determined according to the standard sequence definition of Kabat. Kabat et al. (1987) Sequences of Proteins of Immunological Interest, National Institutes of Health, Bethesda Md. SEQ ID NOs: 1-5 show the heavy chain variable domain sequences of various mouse anti-human IL-23p19 antibodies, and SEQ ID NOs: 9-13 depict the light chain variable domain sequences.

Humanized forms of antibody 13B8 are provided. The humanized light chain 13B8 sequence (with kappa constant region) is provided at SEQ ID NO: 14, and the light chain variable domain comprises residues 1-108 of that sequence. Three versions of the humanized heavy chain 13B8 sequence (with γ1 constant regions) are provided at SEQ ID NOs: 6-8, and the heavy chain variable domain comprises residues 1-116 of those sequences. The 13B8 heavy chains variants are illustrated at Table 1, with differences from the parental sequence noted in bold. The Met (M) was modified to Lys (K) to avoid the potential for oxidation of the residue and inactivation of the antibody. The substitution of AQKLQ for NEMFE is a replacement of the murine CDR sequence with the human germline sequence from the human framework selected to humanize the antibody.

TABLE 1
Antibody 13B8 CDRH2 Variants

| | | SEQ |
|---|---|---|
| Antibody | CDRH2 Sequence | ID NO: |
| m13B8, | QIFPASGSADYNEM | 24 |
| h13B8-a | FEG | |
| h13B8-b | QIFPASGSADYNEK | 25 |
| | FEG | |
| h13B8-c | QIFPASGSADYAQK | 26 |
| | LQG | |

Humanized forms of the other antibodies disclosed herein may be created by simply substituting the parental rodent antibody CDRs into the light and heavy chain sequences for humanized 13B8 provided at SEQ ID NOs: 14 and 6. This approach is most likely to be successful for antibody chains with CDRs having high homology with the CDRs of antibody 13B8, e.g. clone 11C1 on the heavy chain and clones 11C1 and 21D1 on the light chain.

### EXAMPLES

In all of the examples below, frozen droplets of the test compositions were prepared using a metal plate/heat sink apparatus very similar to that shown in FIG. 1. The metal plate/heat sink was made of aluminum and was 10 inches long×7 inches wide×0.4 inches thick and had a flat top surface and a bottom surface with thirty, 1 inch long fins spaced perpendicularly thereto about 0.25 inches apart. The fins were submerged in liquid nitrogen contained in an aluminum reservoir or an STYROFOAM® brand extruded polystyrene foam reservoir that was big enough to hold the metal plate/heat sink.

### Example 1

### Preparation of Lyophilized Spherical Pellets Comprising an IgG1 Antibody

This method of the present invention was exemplified using an IgG1 antibody at 100 mg/ml. A liquid antibody composition comprising the antibody at 100 mg/ml was prepared and frozen droplets of this composition were obtained by pipetting various size aliquots on a solid, flat metal plate having a surface temperature ≦−100° C. Pellets of four different sizes were obtained by aliquoting 20-22 μl, 25 μl, 50 μl and 100 μl of the liquid antibody composition on the cold plate. The frozen droplets were lyophilized and then placed in glass vials for storage. As a control, various volumes (0.25 ml, 0.5 ml, 1 ml and 1.5 ml) of the same liquid antibody composition were placed into 3 ml glass vials and lyophilized. The times required to reconstitute the dried pellets as compared to the same quantity of antibody in dried pellets was measured using a stop watch staring with the addition of a reconstitution volume of SWFI (Sterile Water for Injection) and ending with complete dissolution of all of the dried pellets or lyophilized cake in a glass vial (as determined by visual inspection). Results are shown in Table 2. A configuration listed as “10×20 μl spheres/vial,” for example, refers to a vial containing 10 dried pellets prepared using 20 μl antibody composition. The lyophilized cakes/pellets obtained were also characterized by visual appearance, moisture content analysis and absorbance measurements.

TABLE 2
Reconstitution Times

| | | | Reconstitution | Reconstitution |
|---|---|---|---|---|
| Sample | Configuration | | Volume | Time |
| 1 | 0.25 | ml/vial | 0.25 ml | 3.5 min |
| 2 | 0.5 | ml/vial | 0.5 ml | 4 min |
| 3 | 1.0 | ml/vial | 1 ml | 27 min |
| 4 | 1.5 | ml/vial | 1.5 ml | 16 min |
| 5 | 10 × 20 | μl spheres/vial | 0.2 ml | <1 min |
| 6 | 10 × 20 | μl spheres/vial | 0.2 ml | <1 min |
| 7 | 10 × 25 | μl spheres/vial | 0.25 ml | <1 min |
| 8 | 10 × 25 | μl spheres/vial | 0.25 ml | <1 min |
| 9 | 10 × 50 | μl spheres/vial | 0.5 ml | <1 min |
| 10 | 20 × 50 | μl spheres/vial | 1 ml | <1 min |
| 11 | 20 × 50 | μl spheres/vial | 1 ml | <1 min |
| 12 | 10 × 100 | μl spheres/vial | 1 ml | <1 min |

As is apparent from Table 2, the dried pellets (samples 5-12) were completely dissolved in significantly less time than lyophilized cakes (samples 1-4) containing the same amount of antibody.

### Example 2

### Preparation of Lyophilized Spherical Pellets Comprising an Anti-IL-23p19 Antibody

The method of the present invention was applied to two different liquid formulations of antibody hum13B8-b, a humanized anti-human interleukin-23p19 (anti-IL-23p19) IgG1 monoclonal antibody (mAb). Formulation 1 contained 100 mg/ml of the antibody, 12.5% sucrose, 12.5% trehalose, 0.05% PS-80, 10 mM Histidine, pH 6.0; and Formulation 2 contained 100 mg/ml of the antibody, 7% sucrose, 0.05% PS-80, 10 mM Histidine, pH 6.0). Frozen droplets of these compositions were obtained by dispensing 50 μL liquid at a dispensing speed of 4.5 ml/hr using an automated Biomek® FX dispenser with a 96-pippeting pod onto the solid, flat top surface of the metal plate/heat sink having a surface temperature of −180° C. The frozen droplets were lyophilized on a metal tray and then placed in glass vials for storage. For a control, an aliquot of Formulation 2 was placed in separate 3 cc glass vials and lyophilized.

The lyophilization cycle for frozen beads in the metal tray was annealing at −20° C. for 2 hrs at atmospheric pressure for an hour for followed by a single drying step at 15° C./30 mTorr for 24 hours. In contrast, the lyophilization cycle of Formulation 2 in the control vial employed annealing at −20° C. for 2 hrs at atmospheric pressure, followed by three drying steps: −20° C./100 mTorr for 66.7 hrs, then 5° C./100 mTorr for 1 hr and 30° C./100 mTorr for 7 hrs. The lyophilized pellets and cakes were stored under refrigeration (2-8° C.) for two weeks and then evaluated for solubility and other characteristics relating to antibody stability.

The time required to reconstitute the dried spherical pellets as compared to the same quantity of antibody in dried cake in the control vial was then determined. Four dried pellets from each batch were transferred to a 2 ml type 1 glass vial and 200 microliters of sterile water for injection (SWFI) was added to the vial. The same volume of SWFI was added to the control vial containing dried cake. All of the vials were rotated gently, and the reconstitution time was measured using a stop watch starting with the addition of the SWFI and ending with complete dissolution of all of the dried pellets or lyophilized cake, as determined by visual inspection. As shown in Table 3 below, the reconstitution time of the lyophilized cake was 16 minutes while reconstitution times was <1 min for lyophilized pellets prepared from Formulation 2 and <3 min for lyophilized prepared from Formulation 1, respectively.

Properties of reconstituted solutions prepared from the lyophilized pellets or the cake were characterized by visual inspection, optical density measurement of 100 μl samples at 350 nm, and concentration measurement with a UV-Vis spectrometer. The lyophilized pellets were reconstituted in the same volume of SWFI as the starting volume of the pellets. Since dissolution of pellets causes a small expansion in volume, the total volume after reconstitution was higher compared to the starting volume. The antibody concentration in the reconstituted composition was lower than in the starting composition.

Stability of the antibody in these reconstituted solutions was characterized by high performance ion exchange chromatography (HP-IEX). HP-IEX detects chemical changes in the molecule by separating subpopulations of the same molecules based on their net charge.

Any change in percentage of charged species compared to a reference material is measured. The HP-IEX analysis was performed using a Dionex ProPac® WCX-10 4×250 mm column and mobile phase gradient from 25 mM MES, pH 6, 4% acetonitrile added to 20 mM Phosphate, 95 mM NaCl, pH 8, 4% acetonitrile was added and the UV detection was performed at 280 nm. The results are shown in rows 7-15 of Table 3, with the values in each row representing the percentage of a population of molecules within the sample that have zero net charge at acidic or basic pH with reference to the main peak. No significant differences were observed in the reconstituted solutions prepared from lyophilized pellets or cake.

Aggregate content is a critical quality attribute for biologic drug products. Thus, the aggregate content in the reconstituted solutions was characterized by High Performance Size Exclusion chromatography (HP-SEC), can detect high molecular weight species by separating subpopulations of the same molecules based on their size. The HP-SEC analysis was performed using a YMC-Pack Dial-200 column and a mobile phase of 50 mM Phosphate, 200 mM NaCl, pH 7.0. The results are shown in rows 17-19 of Table 3, with the values in each row representing the percentage of high molecular weight species, monomer or low molecular weight species within the sample. No significant differences were observed in the reconstituted solutions prepared from lyophilized pellets or cake.

The thermal melting profile of the anti IL-23p19 IgG1 antibody in the reconstituted solutions was also characterized using Differential Scanning calorimetry (DSC), with a TA instruments DSC Q2000 V23.10 Build 79 (Tzero pan; TA; Lot#603349; Cat# T110516, Tzero Hermetic lid; TA; Lot#603161; Cat# T110407) used to perform the measurement. Twenty five microliters of reconstituted sample was transferred into the Tzero pan and the pan was sealed. The sample pan and an empty reference pan were placed into the instrument. Sample was equilibrated at 15° C. and then temperature was ramped at 5° C./min to 100° C. Thermal transition temperatures were measured from the enthalpy plot using Universal Analysis software. The results are shown in rows 21-23 of Table 3. As compared to reconstituted solutions of lyophilized pellets or cakes prepared from Formulation 2, the onset melting temperature was slightly increased in the reconstituted solution prepared from lyophilized pellets of Formulation 1, suggesting greater stability of pellets prepared from Formulation 1 under the given experimental condition.

Table 3 provides data characterizing lyophilized pellets prepared from two different formulations containing 100 mg/ml of an anti IL-23p19 IgG mAb and a lyophilized cake prepared from one of these formulations.

TABLE 3
Lyophilized Forms of Anti-IL-23p19 Antibody

| | Formulation 1 | Formulation 2 | Formulation 2 |
|---|---|---|---|
| Appearance | Opaque white | Opaque white | White cake |
| | beads | beads | |
| Reconstitution Time | 2 min 13 sec | 50 sec | 16 min |
| OD 350 nm | 0.065 | 0.090 | 0.083 |
| Concentration | 71.2 mg/ml | 82.7 mg/ml | 90.0 mg/ml |
| HP-IEX | Percentage of a population within the sample | | |
| Acidic Variants | 10.3% | 10.1% | 10.0% |
| Acidic 1 | 8.2% | 8.3% | 8.0% |
| Pre Main | 0.9% | 0.9% | 1.0% |
| Main | 61.2% | 61.5% | 61.7% |
| Post Main | 1.8% | 1.5% | 1.5% |
| Basic 1 | 9.4% | 9.6% | 9.7% |
| Basic 2 | 3.4% | 3.5% | 3.6% |
| Basic Variants | 3.8% | 3.8% | 3.8% |
| Other | 0.8% | 0.8% | 0.8% |
| HP-SEC | Percentage of a population within the sample | | |
| High Molecular | 0.61% | 0.49% | 0.49% |
| Weight Species | | | |
| Monomer | 99.3% | 99.4% | 99.4% |
| Low Molecular | 0.13% | 0.12% | 0.11% |
| Weight Species | | | |
| DSC | Melting Temperature | | |
| Tonset | 71.1° C. | 68.2° C. | 68.3° C. |
| Tm1 | 75.2° C. | 72.7° C. | 73.1° C. |
| Tm2 | 87.9° C. | 86.4° C. | 86.6° C. |

This example illustrates the advantages of lyophilizing a highly concentrated antibody composition in the form of a pellet of substantially spherical shape versus instead of a cake in a vial: a significant reduction in drying and reconstitution times, while still achieving comparable thermostability and biochemical stability, including aggregate content.

### Example 3

### Preparation of Lyophilized Spherical Pellets Comprising a Fusion Protein

The method of the present invention was applied to a liquid composition comprising 25 mg/ml of a TNFRII-Fc fusion protein, which was produced by expression of a recombinant DNA that has a coding sequence for soluble human tumor necrosis factor receptor 2 fused to a coding sequence for the Fc component of human IgG1. The composition also contained 40 mg/ml mannitol, 10 mg/ml sucrose, and 1.2 mg/ml tromethamine in sterile water, pH 7.4.

Droplets of 50 μL each were dispensed using the start/stop function of a KDS Legato™ 200 pump assembled with a 5 ml syringe and 18G needle onto the solid, flat top surface of the metal plate/heat sink apparatus having a surface temperature of ˜−190° C. The frozen droplets were lyophilized in a monolayer format using a lyophilization cycle similar to the Lyophilization Parameters II described above. The lyophilized pellets and cakes were stored under refrigeration (2-8° C.) for two weeks and then evaluated for solubility and other characteristics relating to antibody stability.

To assess the effect of this process on stability of the fusion protein, the dried pellets were reconstituted in 1 ml sterile water, 0.9% benzyl alcohol and thermal unfolding of the fusion protein was measured by Differential Scanning calorimetry (DSC) and Circular Dichroism (CD) spectroscopy. An unlyophilized sample of the same liquid composition was used as a control. CD melts were performed on samples in an auto Peltier 6 cell changer with 1 cm quartz cuvette at a wavelength of 217 nm with a ramp rate of 1 C/min in a temperature range of 20-95° C.

The DSC results indicated that onset temperature for unfolding and mid-point transition temperatures of the fusion protein in the reconstituted formulation was similar to those for the fusion protein in the starting liquid formulation (Tm1 around 77° C. and Tm2 around 88° C. for all the formulations tested). Similarly, the unfolding temperature determined by CD when the signal was measured at 217 nm during temperature ramp was not significantly different between the starting liquid and reconstituted samples (Tm around 65.5° C.).

### Example 4

### Non-Spherical Lyophilized Formulations of Anti-IL-23p19 Antibodies

The present invention discloses methods of preparing lyophilized spherical pellets of antibodies, such as anti-IL-23p19 antibodies, but the formulations presented herein may be used in traditional bulk lyophilization as well in circumstances where that may be preferred. For example, bulk lyophilization may be perfectly acceptable for bulk, long-term storage of drug prior to preparation of a final, packaged commercial drug product. For such purposes, ease and speed of reconstitution may not be important. For such uses, antibody formulations such as Formulation 1 of the present invention may be used in conventional lyophilization procedures, e.g. by the methods described at Example 1 of commonly assigned Int'l Pat. App. Pub. No. WO 2010/027766 A1.

Briefly, filtration, filling, lyophilization, stoppering and capping steps are performed. Filtration involves the following steps. Connect sterilizing filter (0.22 μm) to the sterile receiving vessel. Collect an aliquot of the bulk antibody solution for bioburden testing prior to sterile filtration. Perform aseptic filtration using a 0.22 μm filter into a sterile container. Perform filter integrity testing before and after product filtration.

Filling involves the following steps. Using suitable filling equipment, aseptically fill the antibody product solution into sterilized 13 mm neck, 5 mL, Type I tubing glass vials to achieve a target fill volume of 2.7 ml. Perform fill weight checks during filling. Remove an appropriate number of vials at beginning of filling and pool the solution for bulk sterility and endotoxin testing. Partially seat sterilized lyo-shape stoppers into filled vials. Load the filled vials into a suitable freeze-dryer.

Lyophilization, stoppering and capping involve the following steps. Lyophilize the filled vials using an appropriate lyophilization cycle. After lyophilization is complete, backfill the vials with 0.22 μm filtered nitrogen and fully stopper with 13-mm gray butyl rubber stoppers. Unload the stoppered vials from the lyophilizer and seal them with aluminum crimp seals with polypropylene bonnet. Vials are stored at 2-8° C., and refrigerated when shipped

The resulting vials are inspected for visual defects and stored at 2-8° C. Finished unit dosage vials are shipped under refrigerated conditions.

### Example 5

### Stability of a High Disaccharide Spherical Lyophilized Formulation of Anti-IL-23p19 Antibody

The relative stability of high disaccharide embodiments of the spherical lyophilized formulations of the present invention were determined as follows.

Briefly, antibody hum13B8-b was formulated at 100 mg/ml with either 7% sucrose formulation (7% sucrose, 0.05% polysorbate-80, 10 mM Histidine pH 6.0) or 25% “disaccharide formulation” (12.5% sucrose/12.5% trehalose, 0.05% polysorbate-80, 10 mM Histidine pH 6.0). 1000 μl of the 7% sucrose formulation was lyophilized in a vial as standard cake, whereas the 25% disaccharide formulation was used to form lyospheres (100 μl/bead, 10 beads/vial). The 25% disaccharide formulation lyospheres had faster drying time (24 h versus 80 h) and faster reconstitution times (5 min versus 20 min) compared with the 7% sucrose cake-dried formulation. Sample vials were then stored at either 40° C. or 25° C. for up to 6 months. Samples were reconstituted and analyzed by HP-SEC to detect aggregates. Results are provided at FIG. 6.

Lyospheres made from the 25% disaccharide formulation show enhanced stability compared with standard lyophilized cakes made from the 7% sucrose formulation, with only 0.85% total aggregate after 6 months at 40° C. versus 4.98% aggregate. This increased stability was observed despite the higher moisture content of the lyospheres compared with the lyophilized cakes (˜5.4% versus 0.2%). The 25% disaccharide formulation lyospheres stored at 40° C. were also significantly more stable than the 7% sucrose formulation stored at 25° C.

Table 4 provides a brief description of the sequences in the sequence listing.

TABLE 4
Sequence Identifiers

| SEQ ID NO: | Description |
|---|---|
| 1 | m1A11 V_{H} |
| 2 | m11C1 V_{H} |
| 3 | m5F5 V_{H} |
| 4 | m21D1 V_{H} |
| 5 | m13B8 V_{H} |
| 6 | hum13B8 HC-a |
| 7 | hum13B8 HC-b |
| 8 | hum13B8 HC-c |
| 9 | m1A11 V_{L} |
| 10 | m11C1 V_{L} |
| 11 | m5F5 V_{L} |
| 12 | m21D1 V_{L} |
| 13 | m13B8 V_{L} |
| 14 | hum13B8 LC |
| 15 | m1A11 CDRH1 |
| 16 | m11C1 CDRH1 |
| 17 | m5F5 CDRH1 |
| 18 | m21D1 CDRH1 |
| 19 | m13B8 CDRH1 |
| 20 | m1A11 CDRH2 |
| 21 | m11C1 CDRH2 |
| 22 | m5F5 CDRH2 |
| 23 | m21D1 CDRH2 |
| 24 | m13B8 CDRH2-a |
| 25 | h13B8 CDRH2-b |
| 26 | h13B8 CDRH2-c |
| 27 | m1A11 CDRH3 |
| 28 | m11C1 CDRH3 |
| 29 | m5F5 CDRH3 |
| 30 | m21D1 CDRH3 |
| 31 | m13B8 CDRH3 |
| 32 | m1A11 CDRL1 |
| 33 | m11C1 CDRL1 |
| 34 | m5F5 CDRL1 |
| 35 | m21D1 CDRL1 |
| 36 | m13B8 CDRL1 |
| 37 | m1A11 CDRL2 |
| 38 | m11C1 CDRL2 |
| 39 | m5F5 CDRL2 |
| 40 | m21D1 CDRL2 |
| 41 | m13B8 CDRL2 |
| 42 | m1A11 CDRL3 |
| 43 | m11C1 CDRL3 |
| 44 | m5F5 CDRL3 |
| 45 | m21D1 CDRL3 |
| 46 | m13B8 CDRL3 |
| 47 | human IL-23p19 |
| 48 | mouse IL-23p19 |
| 49 | hum13B8-b HC DNA |
| 50 | hum13B8 LC DNA |
| 51 | ustekinumab CDRH1 |
| 52 | ustekinumab CDRH2 |
| 53 | ustekinumab CDRH3 |
| 54 | ustekinumab CDRL1 |
| 55 | ustekinumab CDRL2 |
| 56 | ustekinumab CDRL3 |
| 57 | ustekinumab V_{H} |
| 58 | ustekinumab V_{L} |
| 59 | ustekinumab HC |
| 60 | ustekinumab LC |
| 61 | briakinumab CDRH1 |
| 62 | briakinumab CDRH2 |
| 63 | briakinumab CDRH3 |
| 64 | briakinumab CDRL1 |
| 65 | briakinumab CDRL2 |
| 66 | briakinumab CDRL3 |
| 67 | briakinumab V_{H} |
| 68 | briakinumab V_{L} |
| 69 | briakinumab HC |
| 70 | briakinumab LC |

1. A method of preparing a lyophilized pellet of an antibody that acts as an antagonist of human IL-23, comprising:
a) providing a vessel which contains a liquid composition comprising the antibody;
b) providing a metal plate comprising a top surface that is solid and flat and a bottom surface that is in physical contact with a heat sink adapted to maintain the top surface of the metal plate at a temperature of −90° C. or below;
c) positioning a dispensing tip above the top surface of the metal plate, the dispensing tip having an open end configured to dispense liquid droplets and another end in fluid contact with the vessel, wherein there is a gap of at least 0.1 cm between the top surface of the metal plate and the open end of the dispensing tip;
d) dispensing an aliquot of the liquid composition through the open end of the dispensing tip as a single droplet onto the top surface of the metal plate in a manner that maintains the droplet as a single droplet having a substantially spherical shape as it contacts and freezes on the top surface; and
e) lyophilizing the frozen droplet to produce a dried pellet of substantially spherical shape.

2. The method of claim 1, wherein the dispensing is performed at a speed and at a gap distance that:
a) prevents freezing of any portion of the aliquot in the tip; and
b) maintains the dispensed droplet in simultaneous contact with the top surface of the metal plate and the open end of the dispensing tip until the droplet surface touching the plate is frozen.

3. The method of claim 2, wherein the dispensing speed is selected from the group consisting of: about 3 ml/min to about 75 ml/min; about 5 ml/min to about 75 ml/min; about 3 ml/min to about 60 ml/min, about 20 ml/min to about 75 ml/min; and about 20 ml/min to about 60 ml/min.

4. The method of claim 2, wherein:
a) the aliquot is 250 μl and the dispensing speed is between about 5 ml/min to about 75 ml/min; or
b) the aliquot is 100 μl and the dispensing speed is between about 3 ml/min to about 60 ml/min; or
c) the aliquot is 20 μl to 50 μl and the dispensing speed is between about 1 ml/min to about 30 ml/min.

5. The method of claim 1, wherein:
a) the top surface temperature of the metal plate is below −150° C.; and
b) the gap distance between the open end of the dispensing tip and the top surface of the metal plate is:
i) between 0.1 cm and 0.5 cm; or
ii) between 0.1 cm and 1 cm; or
iii) between 0.1 cm and 0.75 cm.

6. The method of claim 5, wherein the surface temperature of the metal plate is:
a) between about −180° C. and about −196° C.; or
b) below about −180° C.

7. The method of claim 1, wherein the heat sink comprises a plurality of metal fins having first and send ends and arranged perpendicularly to the metal plate, with a first end of each fin touching the bottom surface of the metal plate and a second end of each fin immersed in liquid nitrogen.

8. The method of claim 1, wherein the liquid composition comprises a total solute concentration of at least 25% on a weight by weight basis.

9. The method of claim 1, wherein the antibody specifically binds to either the p19 subunit of human IL-23 or the IL-23R subunit of the human IL-23 receptor complex.

10. The method of claim 9 wherein the antibody specifically binds to the p19 subunit of human IL-23.

11. The method claim 10 wherein the antibody comprises:
a) an antibody light chain variable domain comprising CDRL1, CDRL2 and CDRL3, wherein:
i) CDRL1 comprises the sequence of SEQ ID NO: 36;
ii) CDRL2 comprises the sequence of SEQ ID NO: 41; and
iii) CDRL3 comprises the sequence of SEQ ID NO: 46,
and
b) an antibody heavy chain variable domain comprising CDRH1, CDRH2 and CDRH3, wherein:
i) CDRH1 comprises the sequence of SEQ ID NO: 19;
ii) CDRH2 comprises a sequence selected from the group consisting of SEQ ID NOs: 24-26; and
iii) CDRH3 comprises the sequence of SEQ ID NO: 31.

12. The method of claim 11 wherein the antibody comprises:
a) an antibody light chain variable domain comprising residues 1-108 of SEQ ID NO: 14; and
b) an antibody heavy chain variable domain comprising a sequence selected from the group consisting of residues 1-116 of SEQ ID NOs: 6-8.

13. The method of claim 12 wherein the antibody comprises:
a) an antibody light chain comprising the sequence of SEQ ID NO: 14; and
b) an antibody heavy chain comprises a sequence selected from the group consisting of SEQ ID NOs: 6-8.

14. The method of claim 1 wherein the antibody specifically binds to the p40 subunit of human IL-23.

15. The method of claim 14 wherein the antibody comprises:
a) an antibody light chain variable domain comprising CDRL1, CDRL2 and CDRL3, wherein:
i) CDRL1 comprises the sequence of SEQ ID NO: 54;
ii) CDRL2 comprises the sequence of SEQ ID NO: 55; and
iii) CDRL3 comprises the sequence of SEQ ID NO: 56,
and
b) an antibody heavy chain variable domain comprising CDRH1, CDRH2 and CDRH3, wherein:
i) CDRH1 comprises the sequence of SEQ ID NO: 51;
ii) CDRH2 comprises the sequence of SEQ ID NO: 52; and
iii) CDRH3 comprises the sequence of SEQ ID NO: 53.

16. The method of claim 15 wherein the antibody comprises:
a) an antibody light chain variable domain comprising the sequence of SEQ ID NO: 58; and
b) an antibody heavy chain variable domain comprising the sequence of SEQ ID NO: 57.

17-24. (canceled)

25. An antibody formulation comprising:
a) an antibody that specifically binds to the p19 subunit of human IL-23;
b) histidine buffer, pH 6.0;
c) sucrose;
d) polysorbate 80; and
e) trehalose.

26. The antibody formulation of claim 25 comprising:
a) 50-120 mg/ml of an antibody that specifically binds to the p19 subunit of human IL-23;
b) about 10 mM histidine buffer, pH 6.0;
c) about 12.5% sucrose;
d) about 0.05% polysorbate 80; and
e) about 12.5% trehalose.

27. (canceled)

28. An antibody formulation comprising:
a) 50-120 mg/ml of an antibody that specifically binds to the p19 subunit of human IL-23;
b) about 10 mM histidine buffer, pH 6.0; and
c) about 0.05% polysorbate 80,
and further comprising:
i) about 30% trehalose;
ii) about 30% sucrose; or
iii) a combination of trehalose and sucrose totaling about 30%.

29. (canceled)

30. The antibody formulation of claim 25 wherein the antibody comprises:
a) an antibody light chain variable domain comprising CDRL1, CDRL2 and CDRL3, wherein:
i) CDRL1 comprises the sequence of SEQ ID NO: 36;
ii) CDRL2 comprises the sequence of SEQ ID NO: 41; and
iii) CDRL3 comprises the sequence of SEQ ID NO: 46,
and
b) an antibody heavy chain variable domain comprising CDRH1, CDRH2 and CDRH3, wherein:
i) CDRH1 comprises the sequence of SEQ ID NO: 19;
ii) CDRH2 comprises a sequence selected from the group consisting of SEQ ID NOs: 24-26; and
iii) CDRH3 comprises the sequence of SEQ ID NO: 31.

31. A lyophilized formulation prepared from the antibody formulation of claim 25.


SEQUENCE LISTING (70 sequences)

SEQ ID NO: 1 - protein, 117 aa, Mus musculus
EVQLQQSGPELVKTGASVNISCKASGYSFTAYYIQWVKQSRGKSLEWIGYISCYNGATRYNQKFKGKATFTVDTSSRTAYMQFSSLTSEDSAVYFCARQGFYAMDYWGQGTSVTVSS

SEQ ID NO: 2 - protein, 116 aa, Mus musculus
HVQLQQSGPEVVRPGASVKLSCKASGYIFSAYWMTWVKQRPGQGLEWIGQIFPVRGSADYNEIFEGKATLTVDTSSSTAYIQLSSLTSEDSAVYYCARGGGGFAYWGQGTLVTVSA

SEQ ID NO: 3 - protein, 124 aa, Mus musculus
QVQLQQSGAELARPGASVKLSCKASGYTFTSYGISWVKQRTGQDLEWIGEIYPRSVNSYYNERFKGKATLTADKSSSTAYMELRSLTSEDSAVYFCARGGNYYGRNYGDYFDYWGQGTTLTVSS

SEQ ID NO: 4 - protein, 116 aa, Mus musculus
QVQLQQSGLELVKPGSSLKISCKASGYSFTSFFIHWLKQRPGQGLEWIGWIFPGNHDVEYNEKFKGKATLTADTSSSTADMHLSSLTSEDSAVYFCARGGGNLPYWGQGTLVTVSA

SEQ ID NO: 5 - protein, 116 aa, Mus musculus
HVQLQQSGPELVRPGASVELSCKASGYIFITYWMTWMKQRPGQGLEWIGQIFPASGSADYNEMFEGKATLTVDTSSNTAYMQLSSLTSEDSAVYFCARGGGGFAYWGQGTLVTVSA

SEQ ID NO: 6 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYNEMFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 446 aa, Artificial Sequence
  Human frameworks, rodent CDRs
QVQLVQSGAEVKKPGASVKVSCKASGYIFITYWMTWVRQAPGQGLEWMGQIFPASGSADYAQKLQGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 113 aa, Mus musculus
NVVMTQTPLTLSVTIGQPASISCKSSQSLLDSDGKTYLNWLLQRPGQSPKRLIYLVSKLDSGVPDRFTGSGSGTDFTLKISRVEAEDLGLYYCWQGTHFPFTFGSGTKLEIKR

SEQ ID NO: 10 - protein, 108 aa, Mus musculus
DIQMTQSPASLSASVGETVTITCRASENIYSYLAWYQEKWGKSPQLLVYNAKTLAEGVPSRFSGSGSGTQFSLKINSLQSEDFGSYYCQHHYGTPFTFGSGTKLEIKR

SEQ ID NO: 11 - protein, 111 aa, Mus musculus
SQAVVTQESALTTSPGETVTLTCRSSTGAVITSNDANWVQEKPDHSFTGLIGGTNNRAPGVPARFSGSLIGDKAALTITGAQTEDEAIYFCALWYSNHWVFGGGTKLTVLG

SEQ ID NO: 12 - protein, 108 aa, Mus musculus
DIQMTQSPVSLSASVGETVTITCRASENVYSYLAWYQQKQGKSPQLLVYNAKTLAEGVPSRFSGSGSGTQFSLKINSLQPEDFGSYYCQHHFGTPLTFGAGTKLDLKR

SEQ ID NO: 13 - protein, 108 aa, Mus musculus
DIQMTQSPASLSASVGETVTITCRTSENIYSYLAWYQQKQGKSPQLLVYNAKTLAEGVPSRFSGSGSGTQFSLKINRLQPEDFGRYFCQHHYGIPFTFGSGTKLEIKR

SEQ ID NO: 14 - protein, 214 aa, Artificial Sequence
  Human frameworks, rodent CDRs
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 10 aa, Mus musculus
GYSFTAYYIQ

SEQ ID NO: 16 - protein, 10 aa, Mus musculus
GYIFSAYWMT

SEQ ID NO: 17 - protein, 10 aa, Mus musculus
GYTFTSYGIS

SEQ ID NO: 18 - protein, 10 aa, Mus musculus
GYSFTSFFIH

SEQ ID NO: 19 - protein, 10 aa, Mus musculus
GYIFITYWMT

SEQ ID NO: 20 - protein, 17 aa, Mus musculus
YISCYNGATRYNQKFKG

SEQ ID NO: 21 - protein, 17 aa, Mus musculus
QIFPVRGSADYNEIFEG

SEQ ID NO: 22 - protein, 17 aa, Mus musculus
EIYPRSVNSYYNERFKG

SEQ ID NO: 23 - protein, 17 aa, Mus musculus
WIFPGNHDVEYNEKFKG

SEQ ID NO: 24 - protein, 17 aa, Mus musculus
QIFPASGSADYNEMFEG

SEQ ID NO: 25 - protein, 17 aa, Artificial Sequence
  Rodent CDR with one amino acid substitution
QIFPASGSADYNEKFEG

SEQ ID NO: 26 - protein, 17 aa, Artificial Sequence
  Rodent CDR with four amino acid substitutions
QIFPASGSADYAQKLQG

SEQ ID NO: 27 - protein, 8 aa, Mus musculus
QGFYAMDY

SEQ ID NO: 28 - protein, 7 aa, Mus musculus
GGGGFAY

SEQ ID NO: 29 - protein, 15 aa, Mus musculus
GGNYYGRNYGDYFDY

SEQ ID NO: 30 - protein, 7 aa, Mus musculus
GGGNLPY

SEQ ID NO: 31 - protein, 7 aa, Mus musculus
GGGGFAY

SEQ ID NO: 32 - protein, 16 aa, Mus musculus
KSSQSLLDSDGKTYLN

SEQ ID NO: 33 - protein, 11 aa, Mus musculus
RASENIYSYLA

SEQ ID NO: 34 - protein, 14 aa, Mus musculus
RSSTGAVITSNDAN

SEQ ID NO: 35 - protein, 11 aa, Mus musculus
RTSENIYSYLA

SEQ ID NO: 36 - protein, 11 aa, Mus musculus
RTSENIYSYLA

SEQ ID NO: 37 - protein, 7 aa, Mus musculus
LVSKLDS

SEQ ID NO: 38 - protein, 7 aa, Mus musculus
NAKTLAE

SEQ ID NO: 39 - protein, 7 aa, Mus musculus
GTNNRAP

SEQ ID NO: 40 - protein, 7 aa, Mus musculus
NAKTLAE

SEQ ID NO: 41 - protein, 7 aa, Mus musculus
NAKTLAE

SEQ ID NO: 42 - protein, 9 aa, Mus musculus
WQGTHFPFT

SEQ ID NO: 43 - protein, 9 aa, Mus musculus
QHHYGTPFT

SEQ ID NO: 44 - protein, 9 aa, Mus musculus
ALWYSNHWV

SEQ ID NO: 45 - protein, 9 aa, Mus musculus
QHHYGIPFT

SEQ ID NO: 46 - protein, 9 aa, Mus musculus
QHHYGIPFT

SEQ ID NO: 47 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 48 - protein, 175 aa, Mus musculus
VPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 49 - DNA, 1398 bp, Artificial Sequence
  Human constant and framework regions, rodent CDRs
atggctgtgctggggctgctgttctgcctggtgacattcccaagctgtgtgctgtcccaggtgcagctggtgcagtctggcgctgaggtgaagaagcctggcgcctccgtgaaggtctcctgcaaggcttctggctacatcttcatcacctactggatgacctgggtgcggcaggcccctggccaggggctggagtggatgggccagatcttccctgccagcggctctgcagactacaacgagaagttcgaaggcagagtcaccatgaccacagacacatccaccagcacagcctacatggagctgaggagcctgagatctgacgacaccgccgtgtattactgtgccagaggcggtggcggattcgcttactggggccagggcaccctggtcaccgtctccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - DNA, 702 bp, Artificial Sequence
  Human constant and framework regions, rodent CDRs
atggctccagtgcagctgctggggctgctggtgctgttcctgccagccatgagatgtgatatccagatgacccagtctccatcctccctgtctgcctctgtgggcgacagagtgaccatcacctgcaggaccagcgagaacatctacagctacctggcctggtatcagcagaagccagggaaggcccctaagctgctgatctataacgccaagaccctggctgaaggggtgccatccaggttcagcggcagcggctctgggacagacttcaccctgaccatcagcagcctgcagcctgaggacttcgccacctactactgtcagcaccactacggaattccattcaccttcggccagggcaccaaggtggagatcaagcgtacggtggctgcaccatctgtgttcatcttccctccatctgatgagcagctgaagtctggaactgcctccgtggtgtgcctgctgaataacttctatcccagagaggccaaggtgcagtggaaggtggataacgccctccagagcggcaactcccaggagagcgtgacagagcaggacagcaaggacagcacctacagcctgagcagcaccctgaccctgagcaaagcagactacgagaaacacaaggtgtacgcctgcgaggtgacccatcagggcctgagcagccccgtgacaaagagcttcaacaggggagagtgttaa

SEQ ID NO: 51 - protein, 5 aa, Homo sapiens
TYWLG

SEQ ID NO: 52 - protein, 16 aa, Homo sapiens
IMSPVDSDIRYSPSFQ

SEQ ID NO: 53 - protein, 10 aa, Homo sapiens
RRPGQGYFDF

SEQ ID NO: 54 - protein, 11 aa, Homo sapiens
RASQGISSWLA

SEQ ID NO: 55 - protein, 7 aa, Homo sapiens
AASSLQS

SEQ ID NO: 56 - protein, 9 aa, Homo sapiens
QQYNIYPYT

SEQ ID NO: 57 - protein, 119 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 58 - protein, 108 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 59 - protein, 449 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 9 aa, Homo sapiens
FTFSSYGMH

SEQ ID NO: 62 - protein, 17 aa, Homo sapiens
FIRYDGSNKYYADSVKG

SEQ ID NO: 63 - protein, 6 aa, Homo sapiens
HGSHDN

SEQ ID NO: 64 - protein, 13 aa, Homo sapiens
SGSRSNIGSNTVK

SEQ ID NO: 65 - protein, 7 aa, Homo sapiens
YNDQRPS

SEQ ID NO: 66 - protein, 12 aa, Homo sapiens
QSYDRYTHPALL

SEQ ID NO: 67 - protein, 115 aa, Homo sapiens [sequence omitted]

SEQ ID NO: 68 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 69 - protein, 445 aa, Homo sapiens
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 217 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS